(19) United States
(12) Patent Application Publication
(10) Pub. No.: US 2008/0159974 A1
(43) Pub. Date: Jul. 3, 2008

(54) Lip plumping cosmetic composition

(76) Inventor: Steve Harripersad, Pompton Lakes, NJ (US)

Correspondence Address: Ward & Olivo, Suite 400, 382 Springfield Avenue, Summit, NJ, 07901, US

(21) Appl. No.: 11/595,231

(22) Filed: Nov. 10, 2006

(51) Int. Cl. A61K 8/18; A61Q 1/04

(52) U.S. Cl. 424/64

(57) ABSTRACT

A cosmetic composition is provided that is particularly intended for application to areas constituting or surrounding the lips. The cosmetic composition contains a viscosity increasing agent as its base, marine collagen, red dye, and a cooling agent. The composition is specifically designed to temporarily enhance the fullness of the lips.

### FIELD OF THE INVENTION

The present invention relates to a cosmetic composition for application to the lips. More specifically, the invention relates to a composition which makes lips plumper and creates the visual and sensory perception that they are fuller. The present invention also reduces facial lines and wrinkles surrounding the lip area.

### BACKGROUND OF THE INVENTION

Many people desire lips that are plumper, fuller and more youthful-looking than the ones they have. There are several products and processes available today that either actually make lips plumper or create the illusion of plumper lips. Some are temporary, some are permanent.

For decades, women have applied lipstick and lip-liner slightly above and below the actual top and bottom of their lips in an attempt to give their lips a fuller appearance. This practice also covers the small facial lines and wrinkles that can surround the mouth as a person ages. In the context of creating fuller lips, lipstick's benefits are that it is temporary and inexpensive—it affords the user maximum flexibility to change the appearance of her lips as often as desired. However, the effect created by lipstick is all an illusion, and not a very natural looking one. Indeed, lipstick cannot be used to create the appearance of fuller lips without imparting a “painted on” look to the user. This prevents lipstick from being utilized to create the appearance of naturally full lips.

In recent years, there has been a proliferation of topical products whose primary function is to plump the lips, create the appearance of fuller lips, or reduce the appearance of fine lines in and around the lips. Many of these products use microencapsulated marine collagen as their plumping ingredient. These microcapsules are directly absorbed into the surface of the lip upon application, providing a temporary infusion of collagen into the lips. Collagen, which is an integral component of skin, provides support for the skin and ensures that it remains pliable and smooth. Over time, however, collagen breaks down forming wrinkles and other indented areas. When collagen is infused into the skin, it fills the indented areas and reinforces the basic structure of the skin which reduces the appearance of wrinkles and provides fuller lips. However, these products do not couple the actual plumping ingredient with other ingredients designed to visually highlight the newly-plumped lips or give the user a sensory experience along with the plumping effect. In other words, the user's lips do “blush” or tingle as the plumping happens.

Injections offer a more semi-permanent way to plump lips and reduce the appearance of the fine lines surrounding the mouth. Arguably the most well-known of these injections is Botox®, but other materials can be used. In order to reduce the appearance of fine lines surrounding the mouth through injection, a material such a Botox® is injected into the area around the lip via needle, inhibiting the contraction of muscles which form wrinkles. As a result, the muscles relax, smoothing out the skin. Alternatively, collagen injections can be used to plump lips. The mechanism of action is the same as the injection of microencapsulated collagen. In other words, it fills the indented areas of skin which form over time and reinforces the basic structure of the skin which reduces the appearance of wrinkles, providing fuller lips. Such injections, however, have several disadvantages. Among other things, they are expensive, invasive, painful, and inconvenient, since they must be administered by a professional.

The most permanent ways to create the appearance of fuller lips or actually achieve plumper lips are through tattooing or surgery. Both of these alternatives have several disadvantages, including their high price, permanence, invasiveness, and risk of infection during the procedure. Tattoos can result in an unnatural look akin to that created by lipstick, and even surgery does not guarantee a natural-looking result.

Thus, it would be advantageous to have a lip plumping composition that is relatively inexpensive, temporary, which does not require injections or surgery for application, and which utilizes one or more ingredients that actually plump the lips in combination with other ingredients that increase the visual and sensory perception of plumpness, all while imparting a look of natural fullness to the user's lips and surrounding area. The present invention is such a composition.

### SUMMARY OF THE INVENTION

The present invention provides a novel means for temporarily providing lips that are fuller. Further, the present invention provides the tactile sensation and appearance of fuller lips. In particular, the invention is a cosmetic composition that is applied to the lips and surrounding area of the mouth in a manner similar to lipstick. In addition, when used on the lips, the invention provides the user with the tactile sensation of “plumping.” This is further enhanced by the fact that the color of the composition is expressed over time, as the tactile sensation increases.

In addition, the cosmetic composition may contain other ingredients designed to soften, moisturize, and smooth the lips in order to minimize the appearance of unsightly lines, as well as prevent additional lines from forming.

The present invention achieves the aforementioned results because it is a chemical composition comprising a viscosity increasing agent as its base, marine collagen, dye, and a cooling agent.

The viscosity increasing agent is preferably polybutene, however, other well known viscosity increasing agents can be utilized as described below.

The marine collagen is preferably atelocollagen, which can be microencapsulated for enhanced efficacy as is known in the art. When collagen is infused into the skin, it fills the indented areas and reinforces the basic structure of the skin which reduces the appearance of wrinkles and provides fuller lips. Other types of marine collagen may also be used.

The cooling agent is preferably menthoxypropanediol, however, other well known cooling agents can be used in accordance with the present invention. The cooling agent in the composition has a dual purpose. First, it stimulates the lips, causing them to plump. Second, it makes the user's lips tingle.

While the preferred composition utilizes red dye, any colored dye can be utilized in accordance with the present invention to add color to the lips. Because the composition is buffered (i.e., pH adjusted), the color develops over time, which provides the visual appearance of fuller lips.

Other optional ingredients can be incorporated into the chemical composition of the present invention. These compounds include preservatives, flavorants, colorants, humectants, hydrocarbons, skin conditioners, occlusives, suspending agents, alcohols, and fats.

In light of the above described disadvantages and insufficiencies of the prior art, it is an object of the invention herein to provide a cosmetic composition for application to the lips to temporarily provide plumper lips.

It is another object of the invention to provide a cosmetic composition for application to the lips and/or surrounding skin area which provides the user with the tactile sensation of plumping lips.

Yet another object of the invention is to provide a visually appealing composition.

Still another object of the invention is to provide a product that is appropriate for use around the mouth.

Yet another object of the invention to provide a product that imparts a desirable feel on the skin.

Yet another object of the invention is to provide an inexpensive lip plumping product.

Yet another object of the invention is to provide a simple method of lip augmentation that is widely affordable.

Yet another object of the invention is to provide a product that plumps the lips which is painless.

Yet another object of the invention is to provide a product that plumps the lips which is temporary.

Other objects, features, and characteristics of the present invention, as well as the methods of operation and functions of the related elements of the structure, and the combination of parts and economies of manufacture, will become more apparent upon consideration of the following detailed description which forms a part of this specification.

### DETAILED DESCRIPTION OF THE PREFERRED EMBODIMENT

A detailed illustrative embodiment of the present invention is disclosed herein. However, the present invention may be embodied in a wide variety of forms, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention.

Moreover, well known methods and procedures for both carrying out the objectives of the present invention and illustrating the preferred embodiment are incorporated herein by reference but have not been described in detail as not to unnecessarily obscure aspects of the present invention.

Initially, the use of the terms “plump(er)”, “full(er)”, and “enhance(d)” is not meant to limit the scope of the present invention. Rather, the terms are used interchangeably for convenience.

As discussed above, the present invention relates generally to a product to temporarily provide both plumper lips and the visual appearance and sensation of fuller lips.

A preferred embodiment of the present invention comprises a composition made from polybutene, marine collagen, red dye, and a cooling agent.

The present invention utilizes polybutene as its base. Polybutene is a well known viscosity increasing agent, and as a result, the composition is viscous when applied to the facial area. The composition is deposited in and around the lips, thereby providing the visual appearance of full lips. It is contemplated that other viscosity increasing agents can be utilized as a base, either alone or in combination with each other. These agents include alkyl galactomannan, silica, talc, magnesium silicate, sorbitol, colloidal silicone dioxide, magnesium aluminum silicate, zinc stearate, wool wax alcohol, sorbiton, sesquioleate, cetyl hydroxy ethyl cellulose and other modified celluloses.

In an alternative embodiment of the present invention, the composition is less viscous than the preferred embodiment. To make a less viscous product, a suitable solvent may be utilized to “thin” the product as is known in the art. Some suitable solvents include propylene glycol, glycerine, cyclomethicone, polyethylene glycols, hexalene glycol, diol and multi-hydroxy based solvents.

The chemical composition of the present invention also utilizes marine collagen. In the preferred embodiment, the marine collagen is atelocollagen. However, other sources of marine collagen can be utilized in accordance with the present invention. When collagen is infused into the skin, it fills the indented areas and reinforces the basic structure of the skin which reduces the appearance of wrinkles and provides fuller lips.

Preferably, the marine collagen is microencapsulated as is known in the art. Microencapsulated marine collagen has increased skin penetration, resulting in a greater concentration of marine collagen delivered to the lips. As a result, the microencapsulated marine collagen provides a greater plumping effect than if it was not microencapsulated.

The composition of the present invention contains pigment to give the composition different shades of color. The preferred embodiment utilizes red dye as a pigment. Because different people have different skin types, and people may prefer a variety of different lip colors, different pigments may be added to different embodiments of the invention. Suitable pigments include various organic and inorganic pigments. The organic pigments are generally aromatic types including azo, indigoid, triphenylmethane, anthraquinone, and xanthine dyes which are designated as D&C and FD&C blues, browns, greens, oranges, yellows, etc. Organic pigments generally consist of insoluble metallic salts of certified color additives, referred to as the Lakes. Inorganic pigments include iron oxides, ultramarines, chromium, chromium hydroxide colors, and mixtures thereof.

In addition, the preferred composition comprises a buffer. The buffer is added to the composition to maintain a specific pH. When properly incorporated into the composition, it acts to suppress the color of the dye when it is initially applied to a user's lips. After it is applied to a user's lips, the system becomes more acidic as it interacts with the natural oils of the lips. This activates the color component of the present composition. Because the composition also contains a cooling agent, the user of the composition both feels and sees the composition plump his or her lips.

In the preferred embodiment, the buffer is citric acid. However, other buffers can be utilized in accordance with the present invention as is known in the art. These compounds include:

The present invention also utilizes a cooling agent in its composition to provide the tactile sensation to the user that his or her lips are plumping. The cooling agent also acts as a skin stimulant, which aids in plumping lips. In the preferred embodiment, the cooling agent is menthoxypropanediol, however, other known cooling agents can be utilized in accordance with the preferred embodiment. Examples include peppermint, spearmint, ginger, and capsaicin. The compositions used in the present invention can contain an oil to permeate and penetrate the skin, providing a moisturizer. The oils can lubricate and soften dry skin as they provide a therapeutic relaxant to tight wrinkled skin. In the preferred embodiment, the composition utilizes mineral oil, pentaerythrityl tetraisostearate, silica dimethyl silyate, sodium chrondoitin sulfate, triisostearyl citrate, silica silyate, castor seed oil, or combinations thereof as moisturizing oils. In alternative embodiments, several nonvolatile oils, volatile oils, and mixtures thereof can be added.

Nonvolatile oils that may be used include such esters as isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, and fatty alcohols such as oleyl alcohol, isocetyl alcohol, and the like.

In another alternative embodiment, the oil may also comprise naturally occurring glyceryl esters of fatty acids, or triglycerides. Both vegetable and animal sources may be used. Examples of such oils include castor oil, lanolin oil, triisocetyl citrate, triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

In yet another alternative embodiment, synthetic or semi-synthetic glyceryl esters can be substituted for oils. For example, fatty acid mono-, di-, and triglycerides which are natural fats or oils that have been modified may be substituted for oils. These oil substitutes include acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, and so on.

Also suitable for use as the oil are nonvolatile hydrocarbons such as isoparaffins, hydrogenated polyisobutene, squalene, petrolatum, and so on.

The compositions may contain a wax to thicken the composition. The wax thickens and stiffens the composition and can possess moisturizing qualities. Alternative embodiments of the invention can include animal waxes, plant waxes, mineral waxes, silicone waxes, synthetic waxes, and petroleum waxes.

Examples of waxes that can be used include bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, stearoxy dimethicone, dimethicone behenate, stearyl dimethicone, and the like, as well synthetic homo- and copolymer waxes from the ethylene series. Preferred waxes are paraffins.

Additional ingredients in the composition can include antioxidants, UV absorbing compounds, preservatives, flavorants, and other materials described herein.

The antioxidants which may be used in the present invention are butylated hydroxyanisole, butylated hydroxytoluene, tocopherol, tocopheryl acetate, ascorbyl palmitate, retinol, retinyl palmitate, hydroquinone and proanthocyanadines.

The ultra-violet absorbing compounds which may be used in the present invention are selected from the group consisting of octyl methoxycimmate, p-aminobenzoic acid, p-aminobenzoic acid esters, benzophenone-3 and other FDA approved sunscreens.

Suitable preservatives which may be utilized include methylparaben, propylparaben, isopropylparaben, isobutylparaben, or combinations thereof.

Optional flavorants which may be utilized include sodium saccharin and other artificial flavors, such as vanilla, The following examples represent preferred compositions.

### EXAMPLE 1

### Preferred Lip Composition Components (By Wt %)

0.01-10% flavorant;
0.01-1.0% red 27 dye;
0.01-1.0% isopropylparaben;
0.01-1.0% isobutylparaben;
0.01-1.0% butylparaben;
0.01-99% mineral oil;
0.01-25% pentaerythrityl tetraisostearate;
0.01-1.0% silica dimethyl silyate;
0.01-1.0% sodium chrondoitin sulfate;
0.01-10% atelocollagen;
0.01-1.0% propylparaben;
0.01-99% polybutene;
0.01-10% triisostearyl citrate;
0.01-50% silica silylate;
0.01-10% menthoxypropanediol;
0.01-1.0% sodium saccharin; and
0.01-1.0% castor seed oil.

### EXAMPLE 2

### Lip Composition (By Wt %)

1.00% flavorant;
0.01% red 27 dye;
0.20% isopropylparaben;
0.15% isobutylparaben;
0.15% butylparaben;
22.15% mineral oil;
5.79% pentaerythrityl tetraisostearate;
0.15% silica dimethyl silyate;
0.038% sodium chrondoitin sulfate;
0.022% atelocollagen;
0.18% propylparaben;
47.124% polybutene;
4.554% triisostearyl citrate;
18.18% silica silylate;
0.20% menthoxypropanediol;
0.04% sodium saccharin; and
0.06% castor seed oil.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof.

What is claimed is:

1. A chemical composition for a cosmetic, wherein said composition comprises:
a viscosity increasing ingredient;
marine collagen;
red dye; and
a cooling agent.

2. A chemical composition according to claim 1, wherein said viscosity increasing agent comprises polybutene.

3. A chemical composition according to claim 1, wherein said viscosity increasing agent is at least one selected from the group consisting of polybutene, alkyl galactomannan, silica, talc, magnesium silicate, sorbitol, colloidal silicone dioxide, magnesium aluminum silicate, zinc stearate, wool wax alcohol, sorbiton, sesquioleate, cetyl hydroxy ethyl cellulose, sorbiton isostearate, and mixtures thereof.

4. A chemical composition according to claim 1, wherein said marine collagen comprises atelocollagen.

5. A chemical composition according to claim 1, wherein said cooling agent comprises menthoxypropanediol.

6. A chemical composition according to claim 1, further comprising at least one skin conditioner.

7. A chemical composition according to claim 6, wherein said skin conditioner comprises at least one selected from the group consisting of pentaerythrityl tetraisostearate, sodium chrondoitin sulfate, triisostearyl citrate, silica silylate, castor seed oil, bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized jojoba oil, synthetic beeswax, synthetic candelilla wax, synthetic carnauba wax, synthetic japan-wax, synthetic jojoba oil, synthetic wax, stearoxy dimethicone, dimethicone behenate, stearyl dimethicone, and synthetic homo- and copolymer waxes from the ethylene series.

8. A chemical composition according to claim 1, further comprising at least one preservative.

9. A chemical composition according to claim 8, wherein said preservative comprises methylparaben, propylparaben, isopropylparaben, isobutylparaben, or combinations thereof.

10. A chemical composition according to claim 8, wherein said preservative comprises at least one selected from the group consisting of butylated hydroxyanisole, butylated hydroxytoluene, tocopherol, tocopheryl acetate, ascorbyl palmitate, retinol, retinyl palmitate, hydroquinone and proanthocyanadines.

11. A chemical composition according to claim 1, wherein said composition comprises nonvolatile hydrocarbons.

12. A chemical composition according to claim 19, wherein said nonvolatile hydrocarbons are selected from the group consisting of: isoparaffins, hydrogenated polyisobutene, mineral oil, squalene, and petrolatum.

13. A chemical composition according to claim 1, wherein said composition further comprises synthetic or semi-synthetic glyceryl esters.

14. A chemical composition according to claim 13, wherein said synthetic or semi-synthetic glycerol esters are selected from the group consisting of: acetylated castor oil, glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, and PEG glyceryl tallowates.

15. A chemical composition according to claim 1, further comprising at least one flavoring agent.

16. A chemical composition according to claim 15, wherein said at least one flavoring agent is selected from the group consisting of alpine vanilla, sodium saccharin.

17. The chemical composition of claim 1, further comprising a buffering agent.

18. The chemical composition of claim 17, wherein said buffering agent comprises citric acid.

19. A cosmetic composition for use on and around the lips comprising:
0.01-10% flavorant;
0.01-1.0% red 27 dye;
0.01-1.0% isopropylparaben;
0.01-1.0% isobutylparaben;
0.01-1.0% butylparaben;
0.01-99% mineral oil;
0.01-25% pentaerythrityl tetraisostearate;
0.01-1.0% silica dimethyl silyate;
0.01-1.0% sodium chrondoitin sulfate;
0.01-10% atelocollagen;
0.01-1.0% propylparaben;
0.01-99% polybutene;
0.01-10% triisostearyl citrate;
0.01-50% silica silylate;
0.01-10% menthoxypropanediol;
0.01-1.0% sodium saccharin; and
0.01-1.0% castor seed oil.

20. A cosmetic composition comprising by weight:
47.124% polybutene;
22.15% mineral oil;
18.18% silica silylate;
5.79% pentaerythrityl tetraisostearate;
4.554% triisostearyl citrate;
1.00% flavorant;
0.20% isopropylparaben;
0.20% menthoxypropanediol;
0.18% propylparaben;
0.15% isobutylparaben;
0.15% butylparaben;
0.15% silica dimethyl silyate;
0.038% sodium chrondoitin sulfate;
0.022% atelocollagen;
0.06% castor seed oil;
0.04% sodium saccharin; and
0.01% red 27 dye.
